(19) United States
(12) Patent Application Publication
(10) Pub. No.: US 2019/0343759 A1
(43) Pub. Date: Nov. 14, 2019

(54) METHOD FOR DELIVERY OF BIOLOGIC AGENTS FROM A TOPICAL FORMULATION

(71) Applicant: Allergan, Inc., Irvine, CA (US)

(72) Inventors: Lance Steward, Irvine, CA (US); Amy Brideau-Andersen, San Clemente, CA (US); Jie Shen, Irvine, CA (US); Lydia Andrews-Jones, Lake Forest, CA (US); Hsiang Ho, Irvine, CA (US); Betsy Bloom, Anaheim, CA (US); Guang Wei Lu, Irvine, CA (US); Fangjing Wang, Irvine, CA (US); Sunny Kumar, Aliso Viejo, CA (US); Gregory Nicholson, Cypress, CA (US); Ron Broide, San Marcos, CA (US)

(21) Appl. No.: 16/408,903

(22) Filed: May 10, 2019

Related U.S. Application Data
(60) Provisional application No. 62/670,711, filed on May 11, 2018.

(51) Int. Cl. A61K 9/00; A61Q 19/08; A61K 8/18; A61K 38/48

(52) U.S. Cl. CPC A61K 9/0014; A61Q 19/08; A61Q 19/007; A61K 38/4893; A61K 8/18

(57) ABSTRACT

Methods for delivery of a biologic agent to the dermis or superficial muscles by topical application of the agent are provided. The methods comprise, in one embodiment, treating or conditioning a skin area to disrupt the stratum corneum in the skin area to define a treated skin area and applying a formulation comprising a biologic agent to the treated skin area.

### CROSS-REFERENCES TO RELATED APPLICATIONS

This application claims the benefit of U.S. Provisional Application No. 62/670,711 filed on May 11, 2018, the entire content of which is incorporated herein by reference.

### TECHNICAL FIELD

The present disclosure relates to a method for delivery of a biologic agent to the dermis and/or superficial muscles from a topically applied formulation.

### BACKGROUND

Human skin is a readily accessible surface for delivery of beneficial agents. Skin of an average adult body covers a surface of approximately 2 m², and receives about one-third of the blood circulating through the body. Skin contains an uppermost layer, epidermis which has morphologically distinct regions; basal layer, spiny layer, stratum granulosum and the upper most stratum corneum. For topical delivery of a beneficial agent to the skin and for transdermal delivery of a beneficial agent to the system, the agent must overcome the barrier properties of the stratum corneum. The stratum corneum is selectively permeable to agents placed on it, and allows only relatively lipophilic compounds with a molecular weight below 400 Daltons to pass across it. Methods of overcoming the barrier properties of the stratum corneum include physical or mechanical methods, such as iontophoresis, skin electroporation, microneedling, and chemical methods, such as penetration enhancers, e.g. dimethylsulfoxide (DMSO), oleyl alcohol, propylene glycol (PG), methyl pyrrolidone, and dodecylazyl cycloheptan 2-one (AZONE®). Such chemical methods may enhance penetration of certain compounds with molecular weights of less than about 500 Daltons, particularly if the molecule is lipophilic or amphiphathic. Hydrophilic compounds, such as proteins and other biologic agents, do not overcome the skin barrier for effective delivery even when known chemical permeation technologies are utilized. Methods that achieve delivery of biologic agents across the skin are desired.

### BRIEF SUMMARY

In one aspect, a method for delivery of a biologic agent is provided. The method comprises treating a skin area to disrupt the stratum corneum in the skin area to define a treated skin area; and applying a formulation to the treated skin area, the formulation comprising the biologic agent and a pharmaceutically acceptable carrier, where the steps of treating and applying achieve topical or transdermal delivery of the biologic agent.

In one embodiment, the treatment comprises a chemical or mechanical treatment or a combination of chemical and mechanical treatments.

In one embodiment, the treatment comprises a formulation comprising hyaluronic acid, wherein a portion of the hyaluronic acid is in crystalline form or microspicule form.

In another embodiment, the treatment is a dermal roller. In one embodiment, the dermal roller has a plurality of microprojections that have a flat, blade-shaped edge that contacts the stratum corneum. In another embodiment, the dermal roller has a plurality of microprojections that have a tapered shaft terminating in a tip that contacts the stratum corneum.

In one embodiment, the skin area is treated with a single pass of the dermal roller. In another embodiment, the skin area is treated with more than a single pass of the dermal roller.

In still another embodiment, the treatment is tape stripping. In one embodiment, the tape stripping comprises applying and removing a strip of tape at least two times. In another embodiment, the tape stripping comprises applying and removing a strip of tape between 2-20 times or between 3-12 times.

In one embodiment, the treating and applying are performed simultaneously.

In some embodiments, the biologic agent is a Clostridial derivative. In one embodiment, the Clostridial derivative is a botulinum toxin. In other embodiments, the biologic agent has a molecular weight of greater than about 100,000 Daltons. In alternative embodiments, the biologic agent has a molecular weight of greater than about 40,000 Daltons.

In another aspect, a method for treating a condition at a localized area with a biologic agent comprises treating a skin area to disrupt the stratum corneum in the skin area to define a treated skin area; and applying a formulation to the treated skin area, the formulation comprising the biologic agent and a pharmaceutically acceptable carrier. The treating and/or applying achieves topical or transdermal delivery of the biologic agent.

In one embodiment, the condition is fine lines or a wrinkle. In one embodiment, the fine lines or wrinkle are selected from the group consisting of lateral canthal lines, glabellar lines, forehead lines, platysma lines, nasolabial lines, perioral lip lines, and combinations thereof.

In other embodiments, the condition is skin laxity. In still other embodiments, the condition is oily skin, sebum, or enlarged pore size. In still other embodiments, the condition is excessive or reduced pigment. In one embodiment, the condition is hyperpigmentation.

In another aspect, a method for improving skin quality is provided. The method comprises treating a skin area to disrupt the stratum corneum in the skin area to define a treated skin area, and applying a formulation to the treated skin area, the formulation comprising a Clostridial derivative and a pharmaceutically acceptable carrier. The treating and/or applying achieves dermal delivery of the Clostridial derivative for an improved skin quality.

In one embodiment, the treating excludes a treating with a chemical solvent, such as but not limited to dimethylsulfoxide (DMSO), oleyl alcohol, propylene glycol (PG), methyl pyrrolidone, and dodecylazyl cycloheptan 2-one (AZONE®).

### BRIEF DESCRIPTION OF THE DRAWINGS

The following drawings are presented to illustrate aspects and features of embodiments of the present methods.

FIG. 1A are images from confocal laser scanning microscopy of human cadaver skin treated with a hyaluronic acid formulation prior to topical application of IgG, fluorescently labelled to visualize the penetration depth of the IgG, the images taken at tissue depths of 0 μm, 8 μm, 16 μm, 24 μm, 32 μm, 40 μm, 48 μm, 56 μm and 64 μm;

FIG. 1B are images from confocal laser scanning microscopy of human cadaver skin with no skin treatment prior to topical application of IgG, fluorescently labelled to visualize the penetration depth of the IgG, the images taken at tissue depths of 0 μm, 8 μm, 16 μm, 24 μm, 32 μm, 40 μm, 48 μm, 56 μm and 64 μm;

FIG. 1C are images from confocal laser scanning microscopy of human cadaver skin treated with a hyaluronic acid formulation and with no IgG, the images taken at tissue depths of 0 μm, 8 μm, 16 μm, 24 μm, 32 μm, 40 μm, 48 μm, 56 μm and 64 μm;

FIG. 2A are images from confocal laser scanning microscopy of human cadaver skin treated with a microneedle dermal roller prior to topical application of IgG, fluorescently labelled to visualize the penetration depth of the IgG, the images taken one hour after IgG application and at 10 μm tissue depths from 0 μm to 280 μm;

FIG. 2B are images from confocal laser scanning microscopy of human cadaver skin with no treatment prior to topical application of IgG, fluorescently labelled to visualize the penetration depth of the IgG, the images taken one hour after IgG application and at 10 μm tissue depths from 0 μm to 280 μm;

FIG. 3A is a bar graph showing the maximum average rat digit abduction score (DAS) observed 3-4 days following intramuscular (IM) or intradermal (ID) injection of BoNT/A (10 U/kg) (n=6 rats/administration method);

FIG. 3B is a bar graph showing the maximum average rat DAS scores after treating the skin with a derma-roller with a needle length of 0.25 mm, 0.5 mm or 1.0 mm, where the roller was passed once (1×) or twice (2×) over the area of skin being treated, and followed by topical application of BoNT/A composition;

FIG. 3C is a bar graph showing the percent of SNAP25₁₉₇-positive staining in neuromuscular junctions of rat tibialis anterior compared to DAS analysis after treating the skin with a derma-roller with a needle length of 0.25 mm, 0.5 mm or 1.0 mm;

FIG. 4 is a bar graph showing the maximum average rat DAS scores after treating the skin with a derma-roller with a needle length of 0.5 mm, where the roller was passed twice over the area of skin being treated, and followed by topical application of two different formulations comprising BoNT/A at the indicated concentrations;

FIG. 5 is a bar graph showing the maximum average rat DAS scores after treating the skin with two different derma-rollers, identified as MTX and DRS, where the roller was passed twice (2×), thrice (3×), four times (4×) or 5 times (5×) over the skin treatment area before applying a topical formulation of BoNT/A;

FIG. 6 is a bar graph showing the mean peak rat DAS scores after treating the skin with a derma-roller before applying a topical formulation comprising a 150 kDa BoNT/A or a 900 kDa BoNT/A; and

FIG. 7 is an immunohistochemistry (IHC) staining showing light SNAP25₁₉₇-positive staining in some motor nerve terminals and axons of rat TA muscle following tape stripping and BoNT/A topical application to the overlying skin surface. No SNAP25₁₉₇ staining was observed in muscles that did not undergo tape stripping.

### DETAILED DESCRIPTION

### Definitions

The following definitions apply herein:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a botulinum toxin to a subject, or alternatively a subject receiving a pharmaceutical composition.

“Alleviating” means a reduction in the occurrence of a condition or symptoms associated with a condition. Thus, alleviating includes some reduction, significant reduction, near total reduction, and total reduction. An alleviating effect may not appear clinically for between 1 to 7 days after administration of a clostridial derivative to a patient or sometimes thereafter.

“Animal protein free” means the absence of blood derived, blood pooled and other animal derived products or compounds. “Animal” means a mammal (such as a human), bird, reptile, fish, insect, spider or other animal species. “Animal” excludes microorganisms, such as bacteria. Thus, an animal protein free pharmaceutical composition can include a botulinum neurotoxin. For example, an “animal protein free” pharmaceutical composition means a pharmaceutical composition which is either substantially free or essentially free or entirely free of a serum derived albumin, gelatin and other animal derived proteins, such as immunoglobulins. An example of an animal protein free pharmaceutical composition is a pharmaceutical composition which comprises or which consists of a botulinum toxin (as the active ingredient) and a suitable polysaccharide as a stabilizer or excipient.

“Biologic agent” intends a molecule that is biologically active and has a molecular weight of 5,000 Daltons or greater, or has a molecular weight in a range of values specified herein.

“Botulinum toxin” means a neurotoxin produced by Clostridium botulinum, as well as a botulinum toxin (or the light chain or the heavy chain thereof) made recombinantly by a non-Clostridial species. The term “botulinum toxin”, as used herein, encompasses Botulinum toxin serotype A (BoNT/A), Botulinum toxin serotype B (BoNT/B), Botulinum toxin serotype C (BoNT/C), Botulinum toxin serotype D (BoNT/D), Botulinum toxin serotype E (BoNT/E), Botulinum toxin serotype F (BoNT/F), Botulinum toxin serotype G (BoNT/G), Botulinum toxin serotype H (BoNT/H), Botulinum toxin serotype X (BoNT/X), and mosaic Botulinum toxins and/or subtypes and variants thereof. “Botulinum toxin”, as used herein, also encompasses a “modified botulinum toxin”. Further “botulinum toxin” as used herein also encompasses a botulinum toxin complex, (for example, the 300, 600 and 900kDa complexes), as well as the neurotoxic component of the botulinum toxin (150 kDa) that is unassociated with the complex proteins.

“Clostridial derivative” refers to a molecule which contains any part of a clostridial toxin. As used herein, the term “clostridial derivative” encompasses native or recombinant neurotoxins, recombinant modified toxins, fragments thereof, a Targeted vesicular Exocytosis Modulator (TEM), or combinations thereof.

“Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/Ci, a BoNT/D, a BoNT/E, a BoNT/F, a BoNT/G, a Tetanus toxin (TeNT), a Baratii toxin (BaNT), and a Butyricum toxin (BuNT). The BoNT/C2 cytotoxin and BoNT/C₃ cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 600-kDa BoNT/A complex, a 300-kDa BoNT/A complex, a 500-kDa BoNT/B complex, a 500-kDa BoNT/Ci complex, a 500-kDa BoNT/D complex, a 300-kDa BoNT/D complex, a 300-kDa BoNT/E complex, and a 300-kDa BoNT/F complex.

“Effective amount” as applied to the biologically active ingredient means that amount of the ingredient which is generally sufficient to induce a desired change in the subject. For example, where the desired effect is a reduction in calculi formation, an effective amount of the ingredient is that amount which causes at least a substantial reduction of bladder overactivity and associated symptoms, and without resulting in significant toxicity.

The term “intact skin” refers to skin that retains its natural barrier function, and has not been altered by chemical means or physical treatment in a way that may harm the barrier function of the stratum corneum. Conversely, “non-intact” skin refers to skin that has been treated in a way that harms the barrier function of stratum corneum.

“Local administration” means administration of a pharmaceutical agent at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient's skin.

“Modified botulinum toxin” means a botulinum toxin that has had at least one of its amino acids deleted, modified, or replaced, as compared to a native botulinum toxin. Additionally, the modified botulinum toxin can be a recombinantly produced neurotoxin, or a derivative or fragment of a recombinantly made neurotoxin. A modified botulinum toxin retains at least one biological activity of the native botulinum toxin, such as, the ability to bind to a botulinum toxin receptor, or the ability to inhibit neurotransmitter release from a neuron or vesicular release from a non-neuronal cells. One example of a modified botulinum toxin is a botulinum toxin that has a light chain from one botulinum toxin serotype (such as serotype A), and a heavy chain from a different botulinum toxin serotype (such as serotype B). Another example of a modified botulinum toxin is a botulinum toxin coupled to a neurotransmitter, such as substance P.

The term “molecular weight” refers to the sum of the atomic weights of all atoms constituting a molecule, and can be numerically expressed in Dalton (Da).

“Mutation” means a structural modification of a naturally occurring protein or nucleic acid sequence. For example, in the case of nucleic acid mutations, a mutation can be a deletion, addition or substitution of one or more nucleotides in the DNA sequence. In the case of a protein sequence mutation, the mutation can be a deletion, addition or substitution of one or more amino acids in a protein sequence. For example, a specific amino acid comprising a protein sequence can be substituted for another amino acid, for example, an amino acid selected from a group which includes the amino acids alanine, asparagine, cysteine, aspartic acid, glutamic acid, phenylalanine, glycine, histidine, isoleucine, lysine, leucine, methionine, proline, glutamine, arginine, serine, threonine, valine, tryptophan, tyrosine or any other natural or non-naturally occurring amino acid or chemically modified amino acids. Mutations to a protein sequence can be the result of mutations to DNA sequences that when transcribed, and the resulting mRNA translated, produce the mutated protein sequence. Mutations to a protein sequence can also be created by fusing a peptide sequence containing the desired mutation to a desired protein sequence.

The term “passive transdermal delivery” refers to delivery of an active agent by placing it on the surface of skin whereby it permeates into the skin as a function of concentration gradient between the higher drug concentration on the skin surface and the lower drug concentration within the skin.

“Peripheral administration” means administration to a location away from a symptomatic location, as opposed to a local administration.

“Pharmaceutical composition” means a composition comprising an active pharmaceutical ingredient, such as, for example, a Clostridial toxin active ingredient such as a botulinum toxin, and at least one additional ingredient, such as, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. It is also envisioned that a pharmaceutical composition comprising a Clostridial toxin active ingredient can include one or more pharmaceutically acceptable excipients that facilitate processing of an active ingredient into pharmaceutically acceptable compositions. Insofar as any pharmacologically acceptable excipient is not incompatible with the Clostridial toxin active ingredient, its use in pharmaceutically acceptable compositions is contemplated. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7th ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

“TEM” as used herein, is synonymous with “Targeted Exocytosis Modulator” or “retargeted endopeptidase” or “Targeted secretion inhibitor (TSI).” Generally, a TEM comprises an enzymatic domain from a Clostridial toxin light chain, a translocation domain from a Clostridial toxin heavy chain, and a targeting domain. The targeting domain of a TEM provides an altered cell targeting capability that targets the molecule to a receptor other than the native Clostridial toxin receptor utilized by a naturally-occurring Clostridial toxin. This re-targeted capability is achieved by replacing the naturally-occurring binding domain of a Clostridial toxin with a targeting domain having a binding activity for a non-Clostridial toxin receptor. Although binding to a non-Clostridial toxin receptor, a TEM undergoes all the other steps of the intoxication process including internalization of the TEM/receptor complex into the cytoplasm, formation of the pore in the vesicle membrane and di-chain molecule, translocation of the enzymatic domain into the cytoplasm, and exerting a proteolytic effect on a component of the SNARE complex of the target cell.

“Topical application” or “topically applying”, as used herein, is meant directly laying or spreading upon epidermal tissue, especially outer skin, where the stratum corneum layer may be intact or disrupted.

“Topical delivery” or “topical administration”, and the like, as used herein mean passage of a topically applied active agent into the skin for localized delivery to the skin.

“Transdermal” as used herein means passage into and/or through skin for localized delivery to superficial muscles or for systemic delivery of an active agent.

“Treating” or “treatment” means to alleviate (or to eliminate) at least one symptom (such as, for example, hip and groin pain), either temporarily or permanently.

“Therapeutically effective amount” refers to an amount sufficient to achieve a desired therapeutic effect.

“Variant” means a Clostridial neurotoxin, such as wild-type botulinum toxin serotype A, B, C, D, E, F, G, H, X, mosaic Botulinum toxins and/or subtypes, hybrids, chimeras thereof that has been modified by the replacement, modification, addition or deletion of at least one amino acid relative to wild-type botulinum toxin, which is recognized by a target cell, internalized by the target cell, and catalytically cleaves a SNARE (SNAP (Soluble NSF Attachment Protein) Receptor) protein in the target cell.

An example of a variant neurotoxin component can comprise a variant light chain of a botulinum toxin having one or more amino acids substituted, modified, deleted and/or added. This variant light chain may have the same or better ability to prevent exocytosis, for example, the release of neurotransmitter vesicles. Additionally, the biological effect of a variant may be decreased compared to the parent chemical entity. For example, a variant light chain of a botulinum toxin type A having an amino acid sequence removed may have a shorter biological persistence than that of the parent (or native) botulinum toxin type A light chain.

### Methods of Treatment

In one aspect, a method for topical or transdermal delivery of a biologic agent is provided. The method comprises treating a skin area to disrupt the stratum corneum in the skin area to define a treated skin area, and applying a formulation to the treated skin area, the formulation comprising a biologic agent and a pharmaceutically acceptable carrier. The treating and/or applying achieves topical or transdermal delivery of the biologic agent. In some embodiments, the treating and/or applying achieve delivery of the biologic agent to, for example, superficial muscle in the treated skin area or to the dermis of the treated skin area. In other aspects, methods for treating a condition at a localized area, for improving skin quality, or for treating damaged or aged skin, are provided. In some embodiments, the biologic agent is a Clostridial derivative.

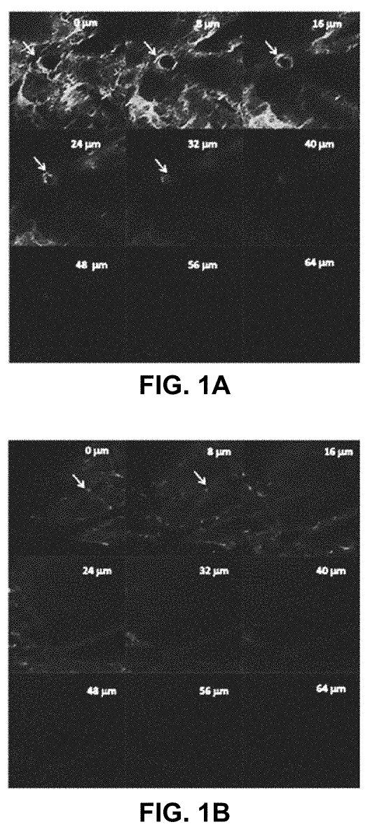

A study was conducted to demonstrate the methods of treating skin conditions by treating a skin area and applying a biologic agent, as described in Example 1. Human cadaver skin was treated with an abrasive scrub containing hyaluronic microcrystals to disrupt the stratum corneum. After treatment with the hyaluronic scrub, a formulation comprising the biologic agent IgG was applied to the treated skin area. The IgG is a 150 kDa protein. It was fluorescently-labeled so that its penetration depth into the skin could be visualized. For comparative controls, skin samples were not treated with the hyaluronic scrub prior to application of the IgG formulation, and other skin samples were treated with the hyaluronic scrub only (i.e., no IgG formulation was applied). The skin samples were scanned at increasing tissue depths with confocal laser scanning microscopy until fluorescence could not be detected and images were captured. Results are shown in FIGS. 1A-1C.

With reference to FIG. 1A, images from confocal laser scanning microscopy of the skin treated with the hyaluronic acid scrub prior to topical application of IgG, are shown at various tissue depths as indicated in each image. The confocal images show that the penetration depth of the fluorescently-labeled IgG was to about 32 μm. The images also show that the morphology of the skin was altered with hyaluronic acid scrub. This is evident by inspection of the images in FIG. 1B, which correspond to the skin control samples that were not treated with hyaluronic scrub. Application of the biologic agent to the skin without treating the skin to disrupt the stratum corneum achieved an IgG penetration depth of about 8 μm. The images of the control sample (FIG. 1B) that received no hyaluronic acid scrub treatment also show that the fluorescence intensity was much lower and nearly invisible. With regard to the other control samples that were treated with the hyaluronic acid scrub and not treated with IgG, the images are shown in FIG. 1C. Very little background fluorescence was detected in the control skin sample treated only with hyaluronic acid scrub. These results demonstrate that the use of an abrasive scrub, such as that containing hyaluronic acid microcrystals, to disrupt the stratum corneum on skin can improve delivery of a biologic agent, such as the 150 kDa IgG, to the epidermal/dermal barrier and shows that large molecule penetration in the skin can be enhanced by disrupting skin structures. In one embodiment, treating the skin to disrupt the stratum corneum in a defined area and applying a formulation with a biologic agent to the treated skin area achieves an increase in penetration depth of the biologic agent into the defined area that is at least about 2, 2.5, 3, 3.5 or 4 times greater than the penetration depth of the biologic agent from the same formulation applied to skin that was not treated to disrupt the stratum corneum.

In another study, a dermal roller was used to treat a skin area to disrupt the stratum corneum. As described in Example 2, human cadaver skin samples were microporated with a dermal roller having needles with a length of 0.5 mm. After treating with the dermal roller, a formulation with fluorescently-labeled IgG was applied to the treated skin area. One hour later, the skin was analyzed with confocal laser scanning microscopy and scanned through skin layers at 10 μm thickness, until fluorescence could be visualized. As a control, skin samples were not treated with the dermal roller prior to applying the IgG formulation.

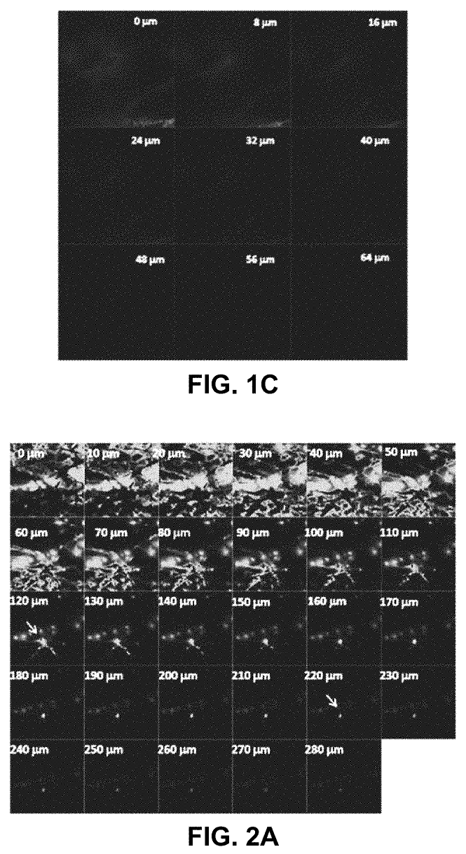

FIG. 2A shows the images of the skin treated with a microneedle dermal roller prior to topical application of fluorescently-labelled IgG. The images show the depth of the created microchannels with 220 μm being a limit for detection of the fluorescently-labelled IgG. FIG. 2B shows the images of the skin that was not treated with the dermal roller prior to topical application of IgG. Fluorescently-labelled IgG could not be visualized beyond about 70 μm depth. These results demonstrated that the biologic agent penetrated three times deeper into human skin after physical treatment using dermal rollers than without physical treatment. The fluorescence marker was visible to approximately 220 μm depth following dermal roller treatment (FIG. 2A) vs. approximately 70 μm depth for control skin with no dermal roller treatment (FIG. 2B). Delivery to 220 μm depth equates to delivery to the dermal layer of skin. These results show that penetration in the skin of a biologic agent with a molecular weight of greater than 5,000 Daltons, 10,000 Daltons, 50,000 Daltons, 75,000 Daltons, or 100,000 Daltons can be enhanced by disrupting skin structures prior to or simultaneously with applying the biologic agent. In one embodiment, the penetration enhancement is at least about 2, 2.5, 3, 3.5, 4, 4.5 or 5 times greater than that obtained for the same biologic agent from the same formulation applied to a similar skin area that was not treated to disrupt the stratum corneum.

Example 3 describes another study where skin was treated to disrupt the stratum corneum to permit delivery of a biologic agent. In this study, the biologic agent was a botulinum neurotoxin type-A (BoNT/A). Skin overlying the tibialis anterior (TA) muscle of rats without hair was microporated with a dermal roller with needle lengths of 0.25 mm, 0.50 mm, or 1.0 mm. The number of passes or the number of times the needle device was rolled over the skin area of treatment was once or twice (i.e., one or two passes, denoted in the drawings as 1×, 2×, etc.). Following treatment with the dermal roller, a formulation comprising a 150 kDa biologic agent, BoNT/A, was applied to the treated skin area.

In order to measure the skin penetration of the (BoNT/A, the rat digit abduction score (DAS) was used. The DAS is a physiological assay that is used to determine the efficacy of BoNT/A on local muscle weakening (Broide, R. S. et al., Toxicon, 71:18-24 (2013)). In brief, following intramuscular (IM) or intradermal (ID) injection, the toxin elicits a dose-dependent reduction in the animal's ability to produce a characteristic hind limb startle response and the degree of this response is scored on a five-point scale. Additionally, the presence of functional BoNT/A in motor nerves within the muscle can be validated by immunohistochemical (IHC) staining for the BoNT/A-cleaved SNAP25 substrate (SNAP25₁₉₇) using a highly selective antibody (Rheaume, C. et al., Toxins (Basel), 7(7), 2354-2370 (2015); Cai, B. B. et al., Neuroscience, 352:155-169 (2017)).

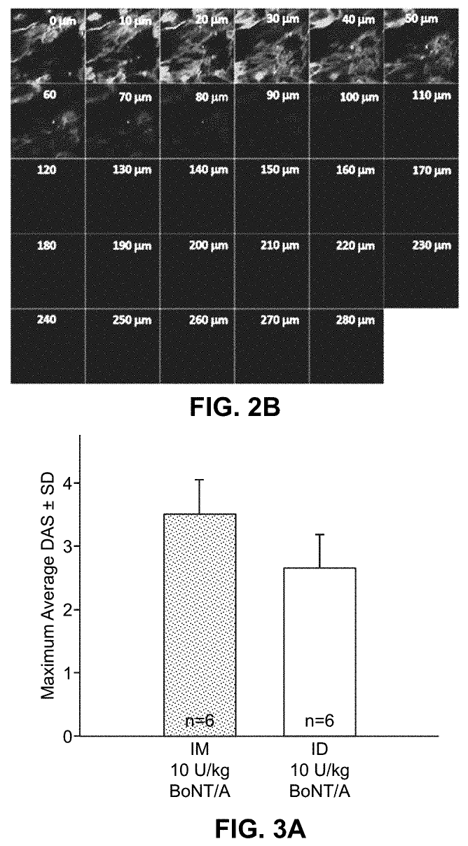

In this study, after topical application, DAS readings were taken on days 1-4 and 7 and the maximum average DAS score for each group was observed and recorded. The scoring scale is set forth in Example 3. An intradermal injection of BoNT/A (10 U/kg; 900 kDa) into the skin overlying the TA muscle was used as a positive control. The intradermal injection has previously been compared to an intramuscular injection at the same toxin dose and the efficacy was found to be only slightly diminished (˜ED70 vs. ˜ED90, respectively), as seen in FIG. 3A.

With reference to FIGS. 3B-3C, microporation with the dermal rollers lead to temporary muscle paralysis in the rats with scores ranging from a DAS of 0 to 4 (maximal response in the assay) (FIG. 3B). Needles with a length of 0.25 mm led to a low DAS score (average <1) with topically applied botulinum neurotoxin. The 0.5 mm and 1.0 mm needle lengths provided substantially better DAS scores (3-4) than the 0.25 mm needle length, demonstrating that the needle length impacts the efficiency of barrier disruption and the resultant efficiency of BoNT/A delivery. Regardless of needle length, two passes with the dermal roller provided increased delivery compared to a single pass.

The rat DAS assay generally involves intramuscular injection of BoNT/A into one of the hindlimb calf muscles, such as the tibialis anterior muscle, followed by DAS scoring. Treatment of the skin tissue overlying the TA muscle of a rat with barrier disruption (e.g., dermal roller) to disrupt the stratum corneum can facilitate delivery of functional BoNT/A from the skin surface to the underlying muscle. As with intramuscular injection, this topical application of BoNT/A also results in a dose-dependent, measurable DAS response.

Quantitative analysis of SNAP25₁₉₇-positive neuromuscular junctions (NMJs) following single dermal roller conditioning (treating) and BoNT/A topical treatment showed a positive correlation with DAS scoring that was dependent on the needle length, as seen in FIG. 3C. As described in Example 3, cross sections of tibialis anterior muscle were double-labeled for 1) the presence of total NMJs using fluorescent-labeled α-bungarotoxin (α-Bgt), which binds to post-synaptic nicotinic acetylcholine receptors (nAChR) and 2) the presence of SNAP25₁₉₇ in the pre-synaptic motor nerve terminals (MNT) and motor nerve (MN) axons using a recombinant monoclonal antibody (Rheaume, C. et al., Toxins (Basel), 7(7), 2354-2370 (2015)). The percentage of SNAP25₁₉₇-positive NMJs out of a representative sampling of total α-Bgt-labeled NMJs throughout the tibialis anterior muscle was calculated and recorded, and the results are in FIG. 3C. The data in FIGS. 3B-3C demonstrate that increased barrier disruption with longer needles and/or more passes over the skin facilitate topical delivery of BoNT/A in skin.

Accordingly, in one embodiment, a method for delivery of a biologic agent to the dermis and/or to superficial muscle is provided. The method comprises treating the skin to disrupt the stratum corneum and applying topically to the treated skin a formulation with the biologic agent. In one embodiment, the biologic agent is delivered to the dermis and/or superficial muscle solely and only by passive transport. Passive transport or diffusion relies on a concentration gradient between the drug at the outer surface and the inner surface of the skin. The diffusion rate is proportional to the gradient and is modulated by a molecule's size, hydrophobicity, hydrophilicity and other physiochemical properties as well as the area of the absorptive surface. In one embodiment, the biologic agent is delivered to the dermis and/or superficial muscle without any active transport. Active transport or delivery relies on, for example, ionization of the biologic agent, or other means to propel the agent into and through the skin. Active transport delivery systems include methods such as iontophoresis, sonophoresis, and thermal microporation.

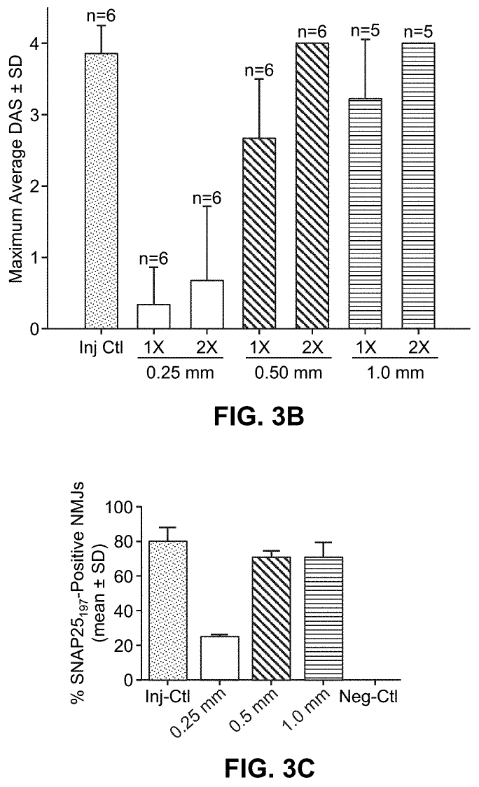

Another study is described in Example 4 where the dose of biologic agent and the formulation on efficiency of delivery into the dermis or superficial muscles following barrier disruption with a dermal roller was evaluated. A skin treatment area in hairless rats was conditioned with a dermal roller having 0.50 mm microneedles by passing the roller over skin area two times. Two formulations comprising BoNT/A at two different doses for each formulation were applied to the treated skin area. Following topical application, DAS readings were taken on days 1-4, and 7 and the maximum average DAS score for each group was determined. FIG. 4 shows the maximum average rat DAS scores for the different formulations. The average peak DAS scores demonstrate that different formulations can deliver BoNT/A with different efficiency when dose, needle length, and number of roller passes are held constant. Regardless of the formulation, increasing topical doses facilitate more efficient delivery of BoNT/A to the TA muscle.

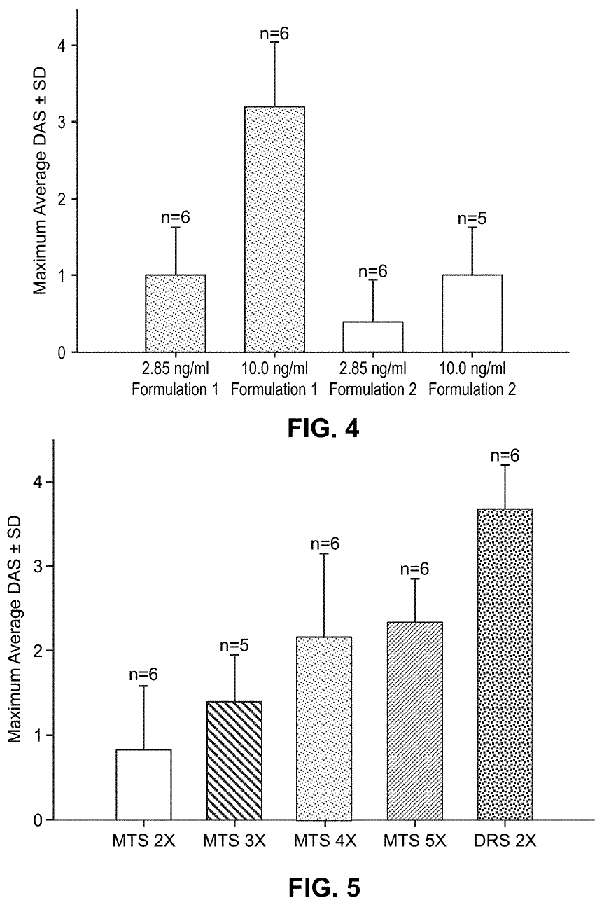

Another study is described in Example 5, where the effect of two different dermal roller on delivery of a biologic agent was evaluated. The dermal rollers each had 0.5 mm needles, with one roller have a higher needle density—with 540 needles (“DRS” roller) versus 200 needles (“MTS” roller). The shape of the needles also differed with one roller having needles with a flatter, blade-like appearance, and the other with cylindrical, tapered needles. A skin treatment area in hairless rats was conditioned with one of the dermal rollers by passing the roller over skin area two times, or for the MTX roller, three times, four times and five times. A formulation comprising BoNT/A was applied to the treated skin area. Following topical application, DAS readings were taken and the maximum average DAS score for each group was determined. FIG. 5 shows the maximum average rat DAS scores for the different roller treatments. The data shows that dermal rollers with different physical designs can be used for delivery of protein biologic agents with a molecular weight of greater than 150,000 Daltons (150 kDa).

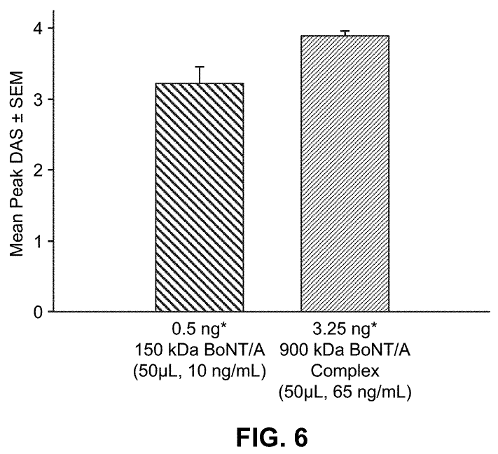

FIG. 6 shows the results of another study that demonstrates delivery of biologic agents with molecular weights of 150,000 Daltons and 900,000 Daltons to the superficial muscle using the methods described herein. A skin area on the rats was conditioned using a dermal roller (Example 6) and after conditioning, a topical formulation with the biologic agent was applied. The DAS assay was used to assess functional delivery of the biologic agent—botulinum toxin—to the muscle. The data in FIG. 6 demonstrate that by combining dermal barrier disruption with topical application, a 900 kDa BoNT/A complex can be delivered as efficiently as a 150 kDa BoNT/A molecule. This was a surprising result given the substantially increased size of the BoNT/A complex compared to purified 150 kDa BoNT/A.

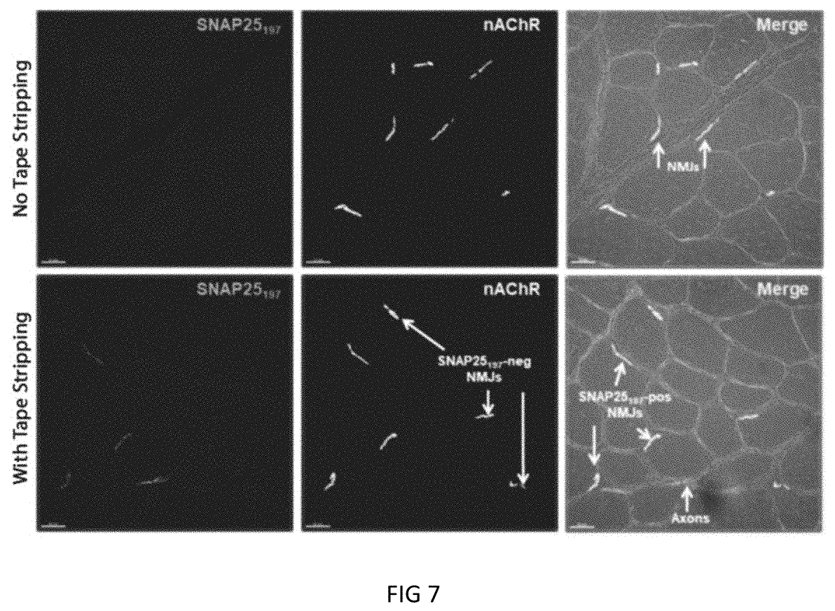

In another study using hairless rats, treating the skin with tape stripping and delivery of a biologic agent to the dermis or underlying superficial muscle was evaluated. As described in Example 7, the skin was tape stripped to disrupt the stratum corneum to facilitate delivery of functional BoNT/A to the underlying muscle. Following pre-treatment with or without the tape stripping, BoNT/A was applied dropwise to the area of skin above the TA muscle. Following topical application, the treated skin and underlying muscle were collected and processed for SNAP25₁₉₇-positive staining by immunohistochemistry and the tissue images are shown in FIG. 7. Following tape stripping and BoNT/A topical application, all superficial muscles showed light SNAP25₁₉₇-positive staining in motor nerve terminals and axons. The percent of SNAP25₁₉₇-labeled NMJs out of a total sampling of NMJs was estimated at ˜20%. No immunohistochemical signal was observed in any muscle that did not undergo tape stripping to the overlying skin surface. These results demonstrate that mild tape stripping is sufficient to facilitate delivery of BoNT/A to the dermis and the superficial muscle layers.

The data in FIGS. 1-7 demonstrate that delivery of a biologic agent to the dermis and/or the superficial muscle is achieved by treating or conditioning the skin to disrupt the stratum corneum and applying a formulation with the biologic agent. The data was generated using human cadaver skin and skin on a hairless rat, as models for human skin. The hairless rat skin has a skin thickness greater than typical rat skin. In the hairless rat, the leg skin surface to panniculus carnosus is about 1.2 mm. In humans, the distance from human face skin surface to cutaneous fat is about 2-3 mm. Observation in the present studies of a positive DAS response demonstrates that barrier disruption facilitates toxin delivery through the hairless rat leg skin to the TA muscle at a distance of greater than ˜1.2 mm (the thickness of rat skin in this area). Therefore, barrier disruption can facilitate toxin delivery to human facial muscles which are 2-3 mm from the skin surface. Botulinum neurotoxin diffuses and spreads in tissues. The degree of diffusion and spread is based on several factors, including dose and volume. Based on the data herein, biologic agents, such as BoNT/A, are able to diffuse 0.5 cm from the epidermis below to the underlying muscle.

As noted above, and based on the data in FIGS. 1-7, methods for delivering a biologic agent to the dermis or superficial muscle are contemplated by conditioning or treating a skin area and applying topically a formulation with the biologic agent to the treated or conditioned skin area. In one aspect, the method is contemplated for treating a skin condition, such as fine lines and/or wrinkles. The fine lines and/or wrinkles can be lateral canthal lines (crow's feet), glabellar lines (vertical lines between the eyebrows), forehead lines, platysma lines (neck lines), nasolabial lines (“smile lines” or “laugh lines) or perioral lip lines (around the mouth and lips). Example 8 provides an example, where a human subject is treated to improve fine lines due to aging.

In other aspects, the methods described herein are contemplated for improving skin quality. The condition or quality of human skin is continuously affected by various factors including, for example, humidity, UV-light, cosmetics, aging, diseases, stress, cigarette smoking, and eating habits, each of which can result in various skin changes. Changes appear on the skin that are characteristic of aging, many of which are reflected by a change in the skin's structure. Some clinical signs of aging of the skin include the appearance of fine lines and deep wrinkles, each of which can increase with age. Wrinkles can be caused by both the chronological aging of the skin and photoaging of skin due to exposure of the skin to sunlight. The method for improving skin quality as contemplated herein includes reversing, slowing the progression of, or preventing skin changes associated with natural aging or the various factors noted above. For example, improving skin quality includes the reversal, slowing the progression of, or prevention of skin changes associated with sun damage or photoaging—i.e., skin changes associated with exposure to sunlight or other forms of actinic radiation (for example, such as UV radiation and tanning booths). As another example, improving skin quality also can include reversing, slowing the progression of, or preventing skin changes resulting from extrinsic factors, including, but not limited to, radiation, air pollution, wind, cold, dampness, heat, chemicals, smoke, cigarette smoking, and combinations thereof. Improving skin quality also can include reversing, preventing or reducing scarring the can result, for example, from certain skin conditions (e.g., acne), infections (i.e., leishmaniasis), or injury (e.g., abrasions, punctures, lacerations, or surgical wounds). Improving skin quality includes decreasing, reducing, and/or minimizing one or more of the skin changes discussed above. Improving skin quality may result in the skin having a more youthful appearance. Improving skin quality may result in the skin having a smoother, hydrated (i.e., less dry), even pigment or less scaly appearance.

With regard to the methods described herein, for improving skin quality, for treating a local skin condition, or for delivery of a biologic agent, the skin contemplated for treatment can include facial skin, skin on the neck, hands, arms, legs, or torso, and skin of other body regions. Skin conditions to be treated or improved include, for example, wrinkles (including, but not limited to, human facial wrinkles), deepening of skin lines, thinning of skin, reduced scarring, yellowing of the skin, mottling, hyperpigmentation, appearance of pigmented and/or non-pigmented age spots, leatheriness, loss of elasticity, loss of recoilability, loss of collagen fibers, abnormal changes in the elastic fibers, deterioration of small blood vessels of the dermis, formation of spider veins, and combinations thereof.

In other embodiments, the method of improving skin quality comprises improving one or more of skin laxity, oily skin, sebum, or enlarged pore size. In still other embodiments, the method is for improving skin quality by treating hyperpigmentation of the skin.

Examples 9-11 describe examples of treating a human subject using the methods contemplated herein to improve skin quality. In Example 9, a human female is treated with a microdermabrasion of a hyaluronic acid microspicule scrub and topical application of a botulinum toxin to the treated skin area, to improve skin quality, including minimizing wrinkles and improving skin laxity. In Example 10, a male human is treated with a dermal roller and with a liquid formulation of BoNT/A to improve skin quality by reducing oiliness, sebum and pore size. In Example 11, a male human is treated with a microdermabrasion of a hyaluronic acid microspicule scrub and topical application of a botulinum toxin to the treated skin area, to improve skin quality by reducing oiliness, sebum and pore size.

As can be appreciated from the examples described, the methods herein comprises a chemical or mechanical treatment or a combination of chemical and mechanical treatments to a skin area. In one embodiment, the treatment comprises a formulation comprising hyaluronic acid, wherein a portion of the hyaluronic acid is in crystalline form or microspicule form. In another embodiment, the treatment is a microneedling device, such as a dermal roller. In one embodiment, the microneedling device has a plurality of microprojections that have a flat, blade-shaped edge that contacts the stratum corneum. In another embodiment, the microneedling device has a plurality of microprojections that have a tapered shaft terminating in a tip that contacts the stratum corneum. In one embodiment, the skin area is treated with a single pass of the microneedling device. In another embodiment, the skin area is treated with more than a single pass of the microneedling device.

In still another embodiment, the treatment is tape stripping. In one embodiment, the tape stripping comprises applying and removing a strip of tape at least two times. In another embodiment, the tape stripping comprises applying and removing a strip of tape between 2-20 times or between 3-12 times.

In one embodiment, the chemical and/or mechanical treatments are performed sequentially or simultaneously. In another embodiment, the chemical and/or mechanical treatments and the applying of the formulation with the biologic agent are performed sequentially or simultaneously.

The biologic agent contemplated for the methods described herein can have a molecular weight of greater than 10 kDa, 25 kDa, 50 kDa, 75 kDa, 100 kDa, 125 kDa, 150 kDa, 175 kDa, 200 kDa, 250 kDa, 300 kDa, 350 kDa, 400 kDa, 500 kDa, 600 kDa, 700 kDa, 800 kDa, 900 kDa, 1,000 kDa, 1,500 kDa, 1,600 kDa, 2,000 kDa, 2,200 kDa, 2,500 kDa, or 3,000 kDa. The biologic agent contemplated for the methods described herein can have a molecular weight of greater than 10 kDa, 25 kDa, 50 kDa, 75 kDa, 100 kDa, 125 kDa, 150 kDa, 175 kDa, 200 kDa, 250 kDa, 300 kDa, 350 kDa, 400 kDa, 500 kDa and less than about 3,000 kDa, 2,500 kDa, 2,200 kDa, 2,000 kDa, 1,600 kDa, 1,500 kDa, or 1,000 kDa.

In one embodiment, the biologic agent is a Clostridial derivative, such as a botulinum neurotoxins (BoNTs), such as, for example, BoNT/A, BoNT/B, etc. These toxins act on the nervous system by blocking the release of neurosecretory substances such as neurotransmitters. The action of BoNT is initiated by its binding to a receptor molecule on the cell surface, and then the toxin-receptor complex undergoes endocytosis. Once inside the cell, BoNT cleaves exocytotic specific proteins responsible for neurotransmitter docking and release from the cell known as the SNARE proteins (soluble N-ethylmaleimide-sensitive factor attachment protein receptor). The resulting transient chemodenervation has been utilized medically to block motor neurotransmission at the neuromuscular junction leading to a variety of therapeutic applications.

In some embodiments, the Clostridial derivative includes a native, recombinant Clostridial toxin, recombinant modified toxin, fragments thereof, TEMs, or combinations thereof. In some embodiments, the Clostridial derivative is a botulinum toxin. In some embodiments, the botulinum toxin can be a botulinum toxin type A, type B, type C₁, type D, type E, type F, type G, H, X, or mosaic Botulinum toxins and/or subtypes, hybrids, chimeras thereof, or any combination thereof. The botulinum neurotoxin can be a recombinantly made botulinum neurotoxins, such as botulinum toxins produced by E. coli. In alternative embodiments, the Clostridial derivative is a TEM.

In some embodiments, the botulinum neurotoxin can be a modified neurotoxin, that is a botulinum neurotoxin which has at least one of its amino acids deleted, modified or replaced, as compared to a native toxin, or the modified botulinum neurotoxin can be a recombinant produced botulinum neurotoxin or a derivative or fragment thereof. In certain embodiments, the modified toxin has an altered cell targeting capability for a neuronal or non-neuronal cell of interest. This altered capability is achieved by replacing the naturally-occurring targeting domain of a botulinum toxin with a targeting domain showing a selective binding activity for a non-botulinum toxin receptor present in a non-botulinum toxin target cell. Such modifications to a targeting domain result in a modified toxin that is able to selectively bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

The Clostridial derivative, such as a botulinum toxin, for use according to the present methods can be stored in lyophilized, vacuum dried form in containers under vacuum pressure or as stable liquids. Prior to lyophilization the botulinum toxin can be combined with pharmaceutically acceptable excipients, stabilizers and/or carriers, such as, for example, albumin, or the like. Acceptable excipients or stabilizers include protein excipients, such as albumin or gelatin, or the like, or non- protein excipients, including poloxamers, saccharides, polyethylene glycol, or the like. In embodiments containing albumin, the albumin can be, for example, human serum albumin or recombinant albumin, or the like. The lyophilized material can be reconstituted with a suitable liquid such as, for example, saline, water, or the like to create a solution or composition containing the botulinum toxin to be administered to the patient.

In some embodiments, the Clostridial derivative is provided in a controlled release system comprising a polymeric matrix encapsulating the Clostridial derivative, wherein a fractional amount of the Clostridial derivative is released from the polymeric matrix over a prolonged period of time in a controlled manner. Controlled release neurotoxin systems have been disclosed for example in U.S. Pat. Nos. 6,585,993; 6,585,993; 6,306,423 and 6,312,708, each of which is hereby incorporated by reference in its entirety.

In alternative embodiments, the Clostridial derivative is provided in an ointment, gel, cream, or emulsion suitable for topical administration.

The therapeutically effective amount of the Clostridial derivative, for example a botulinum toxin, administered according to the present method can vary according to the potency of the toxin and particular characteristics of the pain being treated, including its severity and other various patient variables including size, weight, age, and responsiveness to therapy. The potency of the toxin is expressed as a multiple of the LD₅₀ value for the mouse, one unit (U) of toxin being defined as being the equivalent amount of toxin that kills 50% of a group of 18 to 20 female Swiss-Webster mice, weighing about 20 grams each.

The therapeutically effective amount of the botulinum toxin can vary according to the potency of a particular botulinum toxin, as commercially available botulinum toxin formulations do not have equivalent potency units. It has been reported that one Unit of BOTOX® (onabotulinumA), a botulinum toxin type A available from Allergan, Inc., has a potency Unit that is approximately equal to 3 to 5 Units of DYSPORT® (abobotulinumtoxinA), also a botulinum toxin type A available from Ipsen Pharmaceuticals. MYOBLOC®, a botulinum toxin type B available from Elan, has been reported to have a much lower potency Unit relative to BOTOX®. In some embodiments, the botulinum neurotoxin can be a pure toxin, devoid of complexing proteins, such as XEOMIN® (incobotulinumtoxinA). One unit of incobotulinumtoxinA has been reported to have potency approximately equivalent to one unit of onabotulinumtoxinA. Thus, the quantity of toxin administered and the frequency of its administration will be at the discretion of the physician responsible for the treatment and will be commensurate with questions of safety and the effects produced by a particular toxin formulation.

The dosages used in human therapeutic applications can vary. Typically, the dose of a Clostridial derivative administered to the patient may be up from about 0.01 units to about 1,000 units; for example, up to about 500 units, and preferably in the range from about 80 units to about 460 units per patient per treatment, although smaller or larger doses may be administered in appropriate circumstances.

In some embodiments, the present method comprises administering a composition comprising about 1-500 units of a botulinum toxin type A, such as BOTOX®, to the target site. In some embodiments, the present method comprises administering a composition comprising about 1-100 units of BOTOX® to the target site. In one specific embodiment, the present method comprises administering a composition comprising about 2-50 units of BOTOX® to the target site. In some embodiments, the composition is topically administered to a target site on the face of a subject. In certain embodiments, the dosage can range from about 1 units to about 100 units per treatment. In some embodiments, the dosage per treatment is 2 units, 5 units, 10 units, 20 units, 30 units, 40 units, 50 units, 60 units, 70 units, 80 units, 90 units, 100 units, 110 units, 120 units, 130 units, 140 units, 150 units, 160 units, 170 units, 180 units, 190 units or 200 units of a botulinum toxin type A, such as onabotulinumtoxinA. In alternative embodiments, the present method comprises administering a composition comprising about 3-500 units of abobotulinumA to the target site. In one specific embodiment, the present method comprises administering a composition comprising about 6-250 units of abobotulinumA to the target site. In some embodiments, the composition is topically administered to a target site on the face of a subject. In certain embodiments, the dosage can range from about 3 Units to about 250U per treatment. In yet alternative embodiments, the present method comprises administering a composition comprising about 1-100 units of incobotulinumtoxinA to the target site. In one specific embodiment, the present method comprises administering a composition comprising about 2-50 units of incobotulinumtoxinA to the target site. In some embodiments, the composition is topically administered to a target site on the face of a subject. In certain embodiments, the dosage can range from about 1 unit to about 100 units of incobotulinumtoxinA per treatment. In some embodiments, if the neurotoxin is botulinum toxin type B, the dosage is approximately 50 times greater than the functionally equivalent dosage of botulinum toxin type A.

### EXAMPLES

The following non-limiting examples provide those of ordinary skill in the art with specific preferred methods within the scope of embodiments of the present methods and are not intended to limit the scope thereof.

### Example 1

### Effect of Hyaluronic Acid Scrub Treatment on Skin Penetration of a 150 kDa Biological Agent

Frozen human cadaver skin samples were completely thawed at room temperature (22-25° C.), rinsed briefly with water, placed flat on wet paper towels on a hard board, cleaned using two PBS wet cotton-tipped swabs, and dried with a swab. A hyaluronic acid scrub (MITI Systems, Korea) were applied on the skin evenly, (˜30 mg /˜2×2 cm²) and two drops of mineral oil was added to wet the scrub. The skin with the mixture of oil and scrub was massaged using a gloved finger with moderate pressure for 30 sec, and then wiped using 2 wet cotton-tipped swabs.

FITC-IgG (100 μL of 200 μg/0.5 mL solution) was applied to cover the treated area (˜4 cm²), and the skin was massaged using a gloved finger for 30 sec with moderate pressure. Excess FITC-IgG was removed using 2 wet cotton-tipped swabs. A piece of the treated skin was cut and placed on a glass slide as the treated sample. The same procedure was repeated for two control samples with slight modifications. For the control sample that received no hyaluronic acid scrub treatment, mineral oil without the hyaluronic acid scrub was used massage the skin sample prior to FITC-IgG application. For the control sample without FITC-IgG, hyaluronic acid scrub was applied on the skin followed by application of 100 μL phosphate buffered saline (PBS). The samples were scanned at increasing tissue depths with confocal laser scanning microscopy (CLSM) until fluorescence could not be detected and images were captured. Results are shown in FIGS. 1A-1C.

### Example 2

### Effect of Dermal Roller Treatment on Skin Penetraton of a 150 kDa Biological Agent

Human cadaver skin samples were completely thawed at room temperature (22-25° C.), rinsed briefly with water, and placed flat on wet paper towels on a hard board. The skin was microporated with a microneedling device Dermaroller® MN (MT5 microneedle dermal roller, 0.5 mm) using normal pressure following the guidelines provided by the supplier. The microneedling device was gently rolled 4 times vertically, 4 times horizontally, and then diagonally at the same intensity over the same area 4 times for each direction. A piece of the microporated skin was cut, and 100 μL of FITC-IgG solution (200 μg/0.5 mL) was applied such that it covered the microporated area (at least 1 cm²). The skin was left undisturbed for 60 min in a dark, closed and humidified chamber with wet paper towels, and excess dye was removed using wet saline Kimwipes™ following completion of the incubation period. Dyed micropores were observed with confocal laser scanning microscopy and scanned through skin layers at 10 μm thickness, until no more fluorescence could be visualized. Samples included untreated skin with FITC-IgG and microporated skin with FITC-IgG. Results are shown in FIGS. 2A-2B.

### Example 3

### Effect of Dermal Roller Treatment on Skin Penetration of Functional Botulinum Toxin

The skin overlying the tibialis anterior (TA) muscle of rats without hair was microporated with a microneedling device, a DRS® Derma Roller, with needle lengths of 0.25 mm, 0.50 mm, or 1.0 mm. The number of passes or the number of times the needle device was rolled over the region of interest with the microneedling device was once or twice (i.e., one or two passes, denoted in the drawings as 1×, 2×, etc.). Following pre-treatment with the appropriate microneedling device, 50 μL of 26 ng/mL of 150 kDa BoNT/A in appropriate diluent was then applied dropwise to the skin region of interest along with rubbing/massaging into the tissue. After topical application, DAS readings were taken on days 1-4 and 7 and the maximum average DAS score for each group was observed and recorded. The DAS assay was performed and scored as previously described (Broide, R. S. et al., Toxicon, 71:18-24 (2013)). Briefly, DAS response was induced by grasping the rat around the torso, lifting into the air and simulating a drop or a return to a flat surface. The rat reflexively braces for impact by spreading the digits in its hind paws and the DAS response was immediately scored with the animal facing up in a reclining position. The varying degrees of digit abduction were then scored on a five-point scale (0=normal to 4=maximum reduction in digit abduction). The peak DAS response in a rat is generally observed on days 3-4. An intradermal (ID) injection of BoNT/A (10 U/kg; 900 kDa) into the skin overlying the TA muscle was used as a positive control. The ID injection has previously been compared to an IM injection at the same toxin dose and the efficacy was found to be only slightly diminished (˜ED70 vs. ˜ED90, respectively) (FIG. 3A). Results are shown in FIGS. 3B.

A quantitative analysis of SNAP25₁₉₇-positive neuromuscular junctions (NMJs) following single dermal roller treating) and BoNT/A topical treatment was also done. 14 μm-thick cross sections of TA muscle were double-labeled for 1) the presence of total NMJs using fluorescent-labeled α-bungarotoxin (α-Bgt), which binds to post-synaptic nicotinic acetylcholine receptors (nAChR) and 2) the presence of SNAP25₁₉₇ in the pre-synaptic motor nerve terminals (MNT) and motor nerve (MN) axons using a recombinant monoclonal antibody (Rheaume, C. et al., Toxins (Basel), 7(7), 2354-2370, (2015)). The percentage of SNAP25₁₉₇-positive NMJs out of a representative sampling of total α-Bgt-labeled NMJs throughout the TA muscle was calculated and recorded, and shown in FIG. 3C. Topical application without dermaroller pre-treatment provided no observable DAS score or IHC staining (data not shown).

### Example 4

### Effect of Formulation on Skin Penetration of Functional Botulinum Neurotoxin in the Skin Following Microporation with Dermal Rollers

Hairless rats were pre-treated with a microneedling device to deliver 150 kDa BoNT/A through the skin using a rat DAS model as a read-out for functional transdermal delivery of BoNT/A. A skin treatment area of each rat was primed with a microneedling device, a DRS® Derma Roller with 0.50 mm needles, by passing the roller over the TA muscle twice. The indicated doses of BoNT/A in two different formulations and two different doses for each formulation were applied to the treated skin area. Each test agent was applied in a 50 μL total volume by drop-wise application on the skin above the TA muscle. Following topical application, DAS readings were taken on days 1-4, and 7 and the maximum average DAS score for each group was plotted. The DAS assay was performed and scored as described (Broide, R. S. et al., Toxicon, 71:18-24 (2013)). Peak DAS scores were observed 3-4 days post application of BoNT/A and n=5 or 6 rats per condition evaluated. Results are shown in FIG. 4.

### Example 5

### Analysis of Dermal Rollers on Efficiency of Delivery

Two different microneedling devices were evaluated head-to-head to assess potential differences for delivering BoNT/A across the skin. A DRS50 Derma Roller System device (0.5-mm needle length, 540 needles; China) and an MTS MR5 roller (0.5-mm needle length, 200 needles; Clinical Resolution Laboratories, Inc.) were compared by rolling an area of skin twice with a DRS50 Dermaroller System roller or 2-5 times with an MTS MR5 roller. Given that the DRS50 roller has more than twice the number of needles, the experimental objective was to determine whether an increased number of rolls or passes would be required to achieve comparable penetration with the MR5 roller. It should also be noted that the shape of the needles differs between the two roller types as well. The DRS50 roller has needles with a flatter, blade-like appearance, while the MR5 roller has a more typical cylindrical, tapered needle shape. Results are shown in FIG. 5.

### Example 6

### Efficient Delivery of Functional Botulinum Neurotoxin Complex (900 kDa) in the Skin Following Microporation with Dermal Rollers

Hairless rats were pre-treated with dermarollers to deliver 150 kDa BoNT/A (n=3) or 900 kDa BoNT/A complex (n=6) through the skin using a rat DAS model as a read-out for functional transdermal delivery of BoNT/A. Five passes with an MTS MR5 roller (0.5-mm needle length; Clinical Resolution Laboratories, Inc.) over the TA muscle was conducted prior to the application of a topical test article. Both the 150 kDa BoNT/A and the 900 kDa BoNT/A complex were formulated the same way and the doses were applied to rats following conditioning of the skin with the dermal roller. The formulation with the 150 kDa BoNT/A had a concentration of 10 ng/mL, and was applied in a 50 μL to dose 0.5 ng of BoNT/A. The formulation with the 900 kDa BoNT/A had a concentration of 65 ng/mL, and was applied in a 50 μL to dose 3.25 ng of BoNT/A. The applied dose differences on a mass basis was notable, however, on a molar basis the doses were similar. Mole-wise, slightly more of the 900 kDa complex was dosed. Each test agent was applied in a 50 μL total volume by drop-wise application on the skin above the TA muscle and with rubbing following each drop. Following topical application, DAS readings were taken on days 1-4, and 7 and the maximum average DAS score for each group was plotted. The DAS assay was performed and scored as described above and elsewhere (Broide, R. S. et al., Toxicon, 71:18-24 (2013)). Results are shown in FIG. 6.

### Example 7

### Effect of Tape Stripping on Skin Penetration of Functional Botulinum Neurotoxin

Treatment of hairless rats with tape strips was performed to disrupt the stratum corneum to facilitate delivery of functional BoNT/A the skin surface to the underlying muscle. Rats were tape stripped a few times while a control group received no tape stripping. Following pre-treatment with or without the tape stripping, 50 μL of 7000U/mL (350 U total) of 150 kDa BoNT/A was applied dropwise to the area of skin above the TA muscle. An intradermal injection of 10U/kg 150 kDa BoNT/A was used as a positive control. Following topical application, the treated TA muscles were collected and processed for SNAP25₁₉₇-positive staining by immunohistochemistry as described above. Results are shown in FIG. 7.

### Example 8

### Use of Dermal Roller Barrier Disruption and Topical BoNT to Improve Fine Lines and Laxity

A 45-year female with photo type II skin wants to minimize signs of photo-aged facial skin, including fine lines and laxity. She declines fractional laser treatment to improve the quality of her skin, due to the associated down-time. Instead, she requests topical treatment with a botulinum neurotoxin based product (e.g. BoNT/A). Before treatment, a topical anesthetic cream (2.5% lidocaine and 2.5% prilocaine) is applied on the skin 30 min before treatment and then completely removed with an aseptic wipe.

An approximate 20 cm² area (approximate 5 cm diameter circle) of the face is then primed for treatment by rolling with a 0.5 mm dermal roller 1-10 times vertically, 1-10 times horizontally, 1-10 time diagonally, and 1-10 times on the opposite diagonal. The liquid or liquid reconstituted BoNT/A product (200 μL per 20 cm²) is then immediately applied dropwise onto the primed region with massaging after each drop. The provider gently cleans the face 15 min after the final dropwise application.

Evaluation is conducted at baseline and at 12 weeks' post-treatment. Compared to baseline, at 12 weeks' post-treatment, her facial skin shows higher physician's global assessment and subject satisfaction score, with significant improvement in roughness, hydration, skin elasticity, and trans-epidermal water loss (TEWL).

### Example 9

### Barrier Disruption with an Abrasiva Scrub and Topical Application to Improve Fine Line and Laxity

A 45-year female with photo type II skin wants to minimize signs of photo-aged facial skin, including fine lines and laxity. She declines fractional laser treatment to improve the quality of her skin, due to the associated down-time. Instead, she requests topical treatment with a botulinum neurotoxin based product (e.g. BoNT/A). Before treatment, the skin is cleansed with an aseptic wipe.

An approximate 20 cm2 area (approximate 5 cm diameter circle) of the face is primed by microdermabrasion with a hyaluronic acid microspicule (HA microcrystalline) scrub. 100 mg of HA scrub is applied to the region to be treated and was massaged with gloved fingers for 30 seconds. Any remaining scrub is removed with cotton swabs wetted with sterile water. Liquid BoNT/A or reconstituted BoNT/A product (200 μL per 20 cm2) is then immediately applied topically dropwise onto the primed region with massaging after each drop. The provider gently cleans the face 15 min after the final dropwise application.

Evaluation is conducted at baseline and at 12 weeks' post-treatment. Compared to baseline, at 12 weeks' post-treatment, her facial skin shows higher physician's global assessment and subject satisfaction score, with significant improvement in roughness, hydration, skin elasticity, and trans-epidermal water loss (TEWL).

### Example 10

### Use of Barrier Disruption by Microdermal Roller Followed by Topical BoNT/A to Reduce Oiliness, Sebum and Pore Size

A 35-year male with phototype III skin has oily forehead (seborrhea) and receives topical treatment with BoNT/A.

Before treatment, a topical anesthetic cream (2.5% lidocaine and 2.5% prilocaine) is applied on the skin 30 minutes before treatment and then completely removed with an aseptic wipe. An approximate 20 cm² area (approximate 5 cm diameter circle) of the face is then primed for treatment by rolling with a 0.5 mm dermal roller 1-10 times vertically, 1-10 times horizontally, 1-10 time diagonally, and 1-10 times on the opposite diagonal. The liquid or liquid reconstituted BoNT/A product (200 μL per 20 cm²) is then immediately applied dropwise onto the primed region with massaging after each drop. The provider gently cleans the face 15 min after the final dropwise application.

Evaluation is conducted at baseline and at 12 weeks' post-treatment and the amount of sebum is measured using a sebumeter. Compared to baseline, at 12 weeks' post-treatment, his forehead shows higher physician's global assessment and the patient reported that he was satisfied with the result, with significant reduction in oiliness, sebum and pore size, Percentage (%) reduction in sebum is measured by sebumeter.

### Example 11

### Use of Barrier Disruption with Hyaluronic and Acid Microspicule Scrub Followed By Topical BoNT/A to Reduce Oiliness, Sebum and Pore Size

A 35-year male with phototype III skin has oily forehead (seborrhea) and receives topical treatment with BoNT/A.

Before treatment, the skin is cleansed with an aseptic wipe. An approximate 20 cm² area (approximate 5 cm diameter circle) of the face is then primed by microdermabrasion with a hyaluronic acid micro-spicule (HA microcrystalline) scrub. 100 mg of the scrub is applied to the region to be treated and is massaged with gloved fingers for 30 sec. Any remaining scrub is removed with cotton swabs wetted with sterile water. Liquid BoNT/A or reconstituted BoNT/A product (200 μL per 20 cm²) is then immediately applied dropwise onto the primed region with massaging after each drop. The provider gently cleans the face 15 min after the final dropwise application.

Evaluation is conducted at baseline and at 12 weeks' post-treatment and the amount of sebum is measured using a sebumeter. Compared to baseline, at 12 weeks' post-treatment, his forehead shows higher physician's global assessment and the patient reported that he was satisfied with the result, with significant reduction in oiliness, sebum and pore size, Percentage (%) reduction in sebum is measured by sebumeter.

Many alterations and modifications may be made by those having ordinary skill in the art, without departing from the spirit and scope of the disclosure. Therefore, it must be understood that the described embodiments have been set forth only for the purposes of examples, and that the embodiments should not be taken as limiting the scope of the following claims. The following claims are, therefore, to be read to include not only the combination of elements which are literally set forth, but all equivalent elements for performing substantially the same function in substantially the same way to obtain substantially the same result. The claims are thus to be understood to include those that have been described above, those that are conceptually equivalent, and those that incorporate the ideas of the disclosure.

We claim:

1. A method for delivery of a biologic agent, comprising:
treating a skin area to disrupt the stratum corneum in the skin area to define a treated skin area; and
applying a formulation to the treated skin area, the formulation comprising the biologic agent and a pharmaceutically acceptable carrier,
whereby said treating achieves topical or transdermal delivery of the biologic agent.

2. The method of claim 1, wherein the treating comprises a chemical or mechanical treatment.

3. The method of claim 1, wherein the treatment is a formulation comprising hyaluronic acid, wherein a portion of the hyaluronic acid is in crystalline form.

4. The method of claim 1, wherein the treatment is a dermal roller.

5. The method of claim 4, wherein the dermal roller has a plurality of microprojections that have a flat, blade-shaped edge that contacts the stratum corneum.

6. The method of claim 4, wherein the dermal roller has a plurality of microprojections that have a tapered shaft terminating in a tip that contacts the stratum corneum.

7. The method of claim 1, wherein the treatment is tape stripping.

8. The method of claim 1, wherein the treating and applying are performed simultaneously.

9. The method of claim 1, wherein the biologic agent is a Clostridial derivative.

10. The method of claim 9, wherein the Clostridial derivative is a botulinum toxin.

11. The method of claim 1, wherein the biologic agent has a molecular weight of greater than about 40,000 Daltons.

12. A method for treating a condition at a localized area with a biologic agent, comprising:
treating a skin area to disrupt the stratum corneum in the skin area to define a treated skin area; and
applying a formulation to the treated skin area, the formulation comprising the biologic agent and a pharmaceutically acceptable carrier,
whereby said treating achieves topical or transdermal delivery of the biologic agent.

13. The method of claim 12, wherein the condition is selected from the group consisting of fine lines, a wrinkle, oily skin, sebum, enlarged pore size, and hyperpigmentation.

14. The method of claim 12, wherein the treating comprises a chemical or mechanical treatment.

15. The method of claim 14, wherein the treatment is a formulation comprising hyaluronic acid, wherein a portion of the hyaluronic acid is in crystalline form.

16. The method of claim 14, wherein the treatment is a dermal roller.

17. The method of claim 12, where the treating and applying are performed simultaneously.

18. The method of claim 12, wherein the biologic agent has a molecular weight of greater than about 40,000 Daltons.

19. A method for improving skin quality, comprising:
conditioning a skin area to disrupt the stratum corneum in the skin area to define a conditioned skin area; and
applying a formulation to the conditioned skin area, the formulation comprising a Clostridial derivative and a pharmaceutically acceptable carrier,
whereby said conditioning and applying achieves dermal delivery and/or superficial muscle delivery of the Clostridial derivative.

20. The method of claim 20, where the conditioning and applying are performed simultaneously.
